(19) United States
(12) Patent Application Publication
(10) Pub. No.: US 2018/0099011 A1
(43) Pub. Date: Apr. 12, 2018

(54) Compositions and Methods for Treating Irritable Bowel Syndrome and Related Disorders

(71) Applicant: Crestovo Holdings LLC, Greenwich, CT (US)

(72) Inventor: Thomas Julius BORODY, Five Dock (AU)

(21) Appl. No.: 15/607,004

(22) Filed: May 26, 2017

Related U.S. Application Data
(60) Provisional application No. 62/406,758, filed on Oct. 11, 2016.

(51) Int. Cl. A61K 35/37; A61K 35/74; A61K 9/00; A61K 9/19

(52) U.S. Cl. CPC A61K 35/37; A61K 35/74; A61K 2035/11; A61K 9/0053; A61K 9/19; A61K 9/0031

(57) ABSTRACT

The present disclosure is in the field of pharmaceutical compositions suitable for the treatment of diseases in mammals. The disclosure provides novel compositions comprising non-pathogenic fecal microbes for treating irritable bowel syndrome and related diseases. The disclosure also provides methods for treating a subject with the compositions disclosed herein.

### CROSS-REFERENCE TO RELATED APPLICATIONS

This application is a U.S. Non-Provisional application which claims the benefit of U.S. Provisional Application No. 62/406,758 filed Oct. 11, 2016, which is incorporated by reference in its entirety herein.

### FIELD

The present disclosure relates to pharmaceutical compositions and methods suitable for treating gastrointestinal (GI) disorders, such as irritable bowel syndrome.

### BACKGROUND

Mammals harbor diverse microbial species in their gastrointestinal (GI) tracts. Interactions between these microbes and between microbes and the host, e.g. the host immune system, shape a microbiota. A healthy microbiota provides the host with multiple benefits, including colonization resistance to a broad spectrum of pathogens, essential nutrient biosynthesis and absorption, and immune stimulation that maintains a healthy gut epithelium and an appropriately controlled systemic immunity. An unbalanced microbiota (also called ‘dysbiosis’ or disrupted symbiosis) may lose its function and result in increased susceptibility to pathogens, altered metabolic profiles, or induction of proinflammatory signals that can lead to local or systemic inflammation or autoimmunity. Additionally, such a disrupted microbiota may be infected by incoming pathogen or pathogens, which can cause pain, diarrhea, gas, and constipation among other symptoms. Hence, the intestinal microbiota plays a significant role in the pathogenesis of many disorders such as pathogenic infections of the gut.

Implantation or administration of human colonic microbiota into the bowel of a sick patient is called Fecal Microbiota Transplantation (FMT), also commonly known as fecal bacteriotherapy. FMT is believed to repopulate the gut with a diverse array of microbes that control key pathogens by creating an ecological environment inimical to their proliferation and survival. It represents a therapeutic protocol that allows a fast reconstitution of a normal compositional and functional gut microbial community.

FMT has been used to treat Clostridium difficile infection (CDI). FMT has also been suggested in treating other gut infective agents such as E. coli and Vancomycin resistant Enterococci (VRE). It entails infusions through a colonoscope, an enema or via a nasojejunal tube of human microbiota either in the form of homogenised stool, or cultured stool components such as Clostridia, to implant in the colon and thereby displace or eradicate pathogenic bacteria, e.g., C. difficile.

Irritable bowel syndrome (IBS) is a common disorder affecting the colon. In healthy conditions, muscles of the intestines contract and relax in a rhythmic coordination as food travels from the stomach, through the gastrointestinal tract and to the rectum. In conditions of IBS, the rhythmic contractions and relaxations of the muscles are lost. Contractions can last longer and may be weaker or stronger than normal. The result is IBS with constipation (MS-C), IBS with diarrhea (IBS-D), or IBS with mixed constipation and diarrhea (IBS-M) over periods of hours or days. Existing treatments for irritable bowel syndrome (IBS) involve changes in diet, laxatives, antidiarrheal and anticholinergic drugs with significant side effects. Thus, there is a need for more effective treatments for irritable bowel syndrome that are easier to administer.

### SUMMARY

The present disclosure provides compositions, methods, and dosing regimens for treating or preventing irritable bowel syndrome (IBS).

In one aspect, the present disclosure provides a method for treating irritable bowel syndrome (IBS) in a subject in need thereof, where the method comprises administering to the subject a pharmaceutically active dose of a therapeutic composition comprising or derived from live non-pathogenic fecal bacteria or a sterile fecal filtrate. In one aspect, a sterile fecal filtrate originates from a donor stool. In another aspect, a sterile fecal filtrate originates from cultured microorganisms.

In another aspect, this disclosure provides use of a composition comprising live non-pathogenic fecal bacteria in the manufacture of a medication for the treatment of irritable bowel syndrome.

In one aspect, a method is for treating a form of irritable bowel syndrome selected from the group consisting of irritable bowel syndrome with constipation (IBS-C), irritable bowel syndrome with diarrhea (IBS-D), and irritable bowel syndrome with mixed constipation and diarrhea (IBS-M).

In one aspect, a treatment method achieves at least 50% reduction of constipation in at least 50% patients with IBS-C after 4, 8, or 12 weeks of treatment compared to baseline (e.g., immediately prior to treatment).

In one aspect, a treatment method achieves at least 50% reduction of diarrhea in at least 50% patients with IBS-D after 4, 8, or 12 weeks of treatment compared to baseline (e.g., immediately prior to treatment)

In one aspect, the present disclosure provides a method for treating irritable bowel syndrome (IBS-C, IBS-D, and IBS-M) in a subject in need thereof, where the method comprises administering orally to the subject a pharmaceutically active dose of a therapeutic composition comprising live, non-pathogenic, synthetic bacterial mixture or live, non-pathogenic, purified or extracted, fecal microbiota, where the dose is administered at a dosing schedule of at least once or twice daily or at least once or twice weekly for at least three, eight, ten, or twenty consecutive weeks. In a further aspect, the dose is administered at a dosing schedule of at least once or twice daily or at least one or twice weekly for at least four, five, six, seven, eleven, twelve, thirteen, fourteen, fifteen, sixteen, seventeen, eighteen, or nineteen consecutive weeks.

In one aspect, the present disclosure provides a method for treating irritable bowel syndrome in a subject in need thereof, where the method comprises administering orally to the subject a pharmaceutically active dose of a therapeutic composition comprising a liquid, frozen, lyophilized, or encapsulated sterile fecal filtrate, where the dose is administered at a dosing schedule of at least once or twice daily or at least once or twice weekly for at least three, eight, ten, or twenty consecutive weeks.

In one aspect, a method achieves a remission, cure, response, or resolution rate of irritable bowel syndrome (IBS-C, IBS-D, and IBS-M) of at least about 80%.

In an aspect, a fecal microbiota in a therapeutic composition comprises a donor's substantially entire and non-selected fecal microbiota, reconstituted fecal material, or synthetic fecal material.

### DETAILED DESCRIPTION

Unless defined otherwise herein, terms are to be understood according to conventional usage by those of ordinary skill in the relevant art.

As used herein, the term “treating” refers to (i) completely or partially inhibiting a disease, disorder or condition, for example, arresting its development; (ii) completely or partially relieving a disease, disorder or condition, for example, causing regression of the disease, disorder and/or condition; or (iii) completely or partially preventing a disease, disorder or condition from occurring in a patient that may be predisposed to the disease, disorder and/or condition, but has not yet been diagnosed as having it. Similarly, “treatment” refers to both therapeutic treatment and prophylactic or preventative measures.

As used herein, “therapeutically effective amount” or “pharmaceutically active dose” refers to an amount of a composition which is effective in treating the named disease, disorder or condition.

As used herein, “microbiota,” and “flora” refer to a community of microbes that live in or on a subject's body, both sustainably and transiently, including eukaryotes, archaea, bacteria, and viruses (including bacterial viruses (i.e., phage)). A non-selected fecal microbiota refers to a community or mixture of fecal microbes derived from a donor's fecal sample without selection and substantially resembling microbial constituents and population structure found in such fecal sample.

As used herein, a “sterile fecal filtrate” or a “non-cellular fecal filtrate” refers to a liquid component of a fecal material, where the liquid component is free or substantially free of cell-based living organisms (e.g., bacteria, fungi, or their spores), but retains bacteriophages and non-cellular biological materials. Preferably, a non-cellular or sterile fecal filtrate is also free of viruses for eukaryotic host cells.

As used herein, “remission, cure, or resolution rate” refers to the percentage of patients that are cured or obtain remission or complete resolution of a condition in response to a given treatment. Remission, cure, or resolution of irritable bowel syndrome refers to complete cessation of increased muscle contractions, abdominal cramping, abdominal pain, bloating, gas, diarrhea, constipation, hard stool, dry stool, mucus in the stool, depression, anxiety, fatigue, fibromyalgia, sleep disturbances, and chronic headaches.

As used herein, “response rate” refers to the percentage of patients that respond positively (e.g., reduced severity or frequency of one or more symptoms) to a given treatment. Quantitatively, a patient responds to a treatment positively when the patient's irritable bowel syndrome symptom severity score decreases by at least 2 from baseline to week 8.

As used herein, “irritable bowel syndrome symptom severity score” or “IBS-SSS” refers to an index system for assessing the symptomatic severity or response of an irritable bowel syndrome patient. The index assesses four variables, which include abdominal pain, abdominal distension (e.g., bloating), bowel habits, and quality of life. See Francis et al., The irritable bowel severity scoring system: a simple method of monitoring irritable bowel syndrome and its progress. Aliment Parmacol Ther. 1997; 11:395-402. Each variable is scored from 0-100 so that the total index score ranges from 0-500; 0-75: remission; 75-175: mild; 175-300: moderate; >300: severe irritable bowel syndrome.

As used herein, “eukaryotic” refers to belonging to a cell that contains a nucleus and membrane-bound organelles.

As used herein, “bacteria,” “bacterium,” and “archaea” refer to single-celled prokaryotes that lack membrane bound nuclei and lack organelles.

As used herein, “colony forming units” (cfu) refers to an estimate of the number of viable microorganism cells in a given sample.

As used herein, “viable” means possessing the ability to multiply.

As used herein, “fecal bacteria” refers to bacteria that can be found in fecal matter.

As used herein, “isolated” or “purified” refers to a bacterium or other entity or substance that has been (1) separated from at least some of the components with which it was associated when initially produced (whether in nature or in an experimental setting), and/or (2) produced, prepared, purified, and/or manufactured by the hand of man. Isolated or purified bacteria can be separated from at least about 10%, about 20%, about 30%, about 40%, about 50%, about 60%, about 70%, about 80%, about 90%, or more of the other components with which they were initially associated.

As used herein, “cytotoxic” activity or bacterium includes the ability to kill a bacterial cell, such as a pathogenic bacterial cell. A “cytostatic” activity or bacterium includes the ability to inhibit, partially or fully, growth, metabolism, and/or proliferation of a bacterial cell, such as a pathogenic bacterial cell.

As used herein, the terms “pathogen” and “pathogenic” in reference to a bacterium or any other organism or entity includes any such organism or entity that is capable of causing or affecting a disease, disorder or condition of a host organism containing the organism or entity.

As used herein, “spore” or a population of “spores” includes bacteria (or other single-celled organisms) that are generally viable, more resistant to environmental influences such as heat and bacteriocidal agents than vegetative forms of the same bacteria, and typically capable of germination and out-growth. “Spore-formers” or bacteria “capable of forming spores” are those bacteria containing the genes and other necessary abilities to produce spores under suitable environmental conditions.

As used herein, a “combination” of two or more bacteria includes the physical co-existence of the two bacteria, either in the same material or product or in physically connected products, as well as the temporal co-administration or co-localization of the two bacteria.

As used herein, “subject” refers to any animal subject including humans, laboratory animals (e.g., primates, rats, mice), livestock (e.g., cows, sheep, goats, pigs, turkeys, chickens), and household pets (e.g., dogs, cats, rodents, etc.). The subject or patient may be healthy, or may be suffering from an infection due to a gastrointestinal pathogen or may be at risk of developing or transmitting to others an infection due to a gastrointestinal pathogen.

As used herein, “Shannon Diversity Index” refers to a diversity index that accounts for abundance and evenness of species present in a given community using the formula

$$H = - {\sum }_{i = 1}^{R} {p}_{i} ln {p}_{i}$$

where H is Shannon Diversity Index, R is the total number of species in the community, and pᵢ is the proportion of R made up of the ith species. Higher values indicate diverse and equally distributed communities, and a value of 0 indicates only one species is present in a given community. For further reference, see Shannon and Weaver, (1949) The mathematical theory of communication. The University of Illinois Press, Urbana. 117pp.

As used herein, “antibiotic” refers to a substance that is used to treat and/or prevent bacterial infection by killing bacteria, inhibiting the growth of bacteria, or reducing the viability of bacteria.

As used herein, an “intermittent dosing schedule” means that that a therapeutic composition is administered for a period of time followed by a period of time (a treatment period) where treatment with such therapeutic composition is withheld (a rest period). Intermittent dosing regimens can be expressed as treatment period in days or weeks/rest period in days or weeks. For example, a 4/1 intermittent dosing schedule refers to an intermittent dosing schedule where the treatment period is four weeks/days and the rest period is one week/day.

As used herein, a “continuous dosing schedule” refers to a dosing schedule where a therapeutic composition is administered during a treatment period without a rest period. Throughout the treatment period of a continuous dosing schedule, a therapeutic composition can be administered, for example, weekly, daily, or every other day, or every third day. On a day when a therapeutic composition is administered, it can be administered in a single dose, or in multiple doses throughout the day.

As used herein, “dosing frequency” refers to the frequency of administering doses of a therapeutic composition in a given time. Dosing frequency can be indicated as the number of doses per a given time, for example, once per day, once a week, or once in two weeks.

As used herein, “dosing interval” refers to the amount of time that elapses between multiple doses being administered to a subject.

Without being bound to any theory, irritable bowel syndrome is considered by some as a chronic and common disorder affecting the colon. In healthy conditions, muscles of the intestines contract and relax in a rhythmic coordination as food travels from the stomach, through the gastrointestinal tract and to the rectum. In conditions of irritable bowel syndrome, the rhythmic contractions and relaxations of the muscles are lost. Contractions can last longer and may be weaker or stronger than normal. The result is irritable bowel syndrome with symptoms of constipation or diarrhea, or both constipation and diarrhea. A variety of factors may lead to irritable bowel syndrome, including food allergies or intolerance, hormones, and stress. Bacterial overgrowth as a result of other illnesses that affect the GI tract can also lead to irritable bowel syndrome.

Irritable bowel syndrome (IBS) is characterized by recurrent abdominal discomfort or pain that is accompanied by at least two of the following: relief by defecation, change in frequency of stool, or change in appearance of stool. Changes in normal intestinal muscle contractions cause symptoms of abdominal cramping or pain, bloating, gas, diarrhea or constipation, hard and dry stool, and mucus in the stool. Additional symptoms can include depression, anxiety, fatigue, fibromyalgia, sleep disturbances, and chronic headaches. Other digestive tract disorders, including appendicitis, gallbladder disease, ulcers, and cancer can also develop in a subject with irritable bowel syndrome.

Treatment of IBS varies depending on the severity of the syndrome and focuses on the relief of symptoms. Mild cases of IBS can be treated by decreasing stress and avoiding foods known to trigger symptoms. Anti-depressants are prescribed in both mild and severe cases. In severe cases, anti-diarrheal medications and fiber supplements may be used to control diarrhea and constipation, respectively. If symptoms are due to abnormal gut flora or bacteria overgrowth, treatment can include probiotics and antibiotics. IBS specific medications prescribed in severe cases include Alosetron, Rifaximin, Eluxadoline, Linaclotide and Lubiprostone. Alosetron slows the movement of bowels by relaxing the colon muscles. Lubiprostone aids patients with IBS-C by increasing fluid secretion of the small intestines.

While the etiology of irritable bowel syndrome is unknown, emotional factors, diet, drugs, or hormones are believed to aggravate the GI symptoms. Subjects with IBS can also have anxiety disorders, depression, or a somatization disorder. Physiologic factors, including altered motility, visceral hyperalgesia, and various environmental and genetic factors, can also play a role in IBS.

Irritable bowel syndrome is the most frequently diagnosed gastrointestinal disorder. This syndrome affects 10 to 20% of the general population. Women are twice as likely to suffer from IBS than men. However, women are also considered by some to be more likely to consult a doctor.

Irritable bowel syndrome occurs most often in people ages 15 to 30, although the disease may afflict people of any age. It affects men and women equally and appears to run in some families.

Different types of irritable bowel syndrome exist. As used herein, “irritable bowel syndrome with constipation” or “IBS-C” refers to a form of irritable bowel syndrome accompanied with a decrease in stool frequency.

As used herein, “irritable bowel syndrome with diarrhea” or “IBS-D” refers to a form of irritable bowel syndrome accompanied with increased stool frequency.

As used herein, “irritable bowel syndrome mixed type” or “IBS-M” refers to a form of irritable bowel syndrome accompanied with alternating increases and decreases in stool frequency.

Many chronic diseases and disorders of the GI tract have chronic infection/infestation as their underlying pathological conditions (e.g., irritable bowel syndrome). In one aspect, the present disclosure includes and relates to the use of a fecal microbiota, one or more microbial species therefrom, an active fragment or component therefrom for the treatment and/or prophylaxis of various disease states (e.g., irritable bowel syndrome) related to the presence of ‘abnormal’ microflora in the GI tract. An active fragment of a bacterium can be any active molecule isolated from such bacteria by any known method for preparing/identifying active fragments of bacteria and proteins secreted from bacteria. Such methods include but are not limited to the following: sonication, osmotic shock, detergent lysis, high pressure, transfer appropriate DNA to other organisms, such as bacteria, plant or animal that is then used as a feed additive as described previously. In one aspect, an active fragment or component of a bacterium is selected from the group consisting of a mycolate or a derivative thereof, a polysaccharide, a lipoglycan, a small peptide, a thiopeptide, a protein, a nucleic acid molecule, a metabolite, a cell wall component, or any combination thereof. In one aspect, an active fragment is a protein or a secretion. In another aspect, an active fragment is a secreted protein.

In one aspect, the present disclosure provides a method for treating irritable bowel syndrome in a subject in need thereof, where the method comprises administering to the subject a pharmaceutically active dose of a therapeutic composition comprising live non-pathogenic fecal bacteria. In another aspect, this disclosure provides use of a composition comprising live non-pathogenic fecal bacteria in the manufacture of a medication for the treatment of irritable bowel syndrome.

In one aspect, a method provided here is for treating a form of irritable bowel syndrome selected from the group consisting of IBS-C, IBS-D, and IBS-M. In one aspect, a therapeutic composition comprises an isolated or purified population of live non-pathogenic fecal bacteria. In one aspect, a therapeutic composition comprises a non-selected fecal microbiota. In another aspect, a therapeutic composition comprises a non-selected and substantially complete fecal microbiota. In another aspect, a therapeutic composition comprises a full-spectrum fecal microbiota. In one aspect, a method further comprises administering a 5-aminosalicylic acid agent, a corticosteroid, an immunosuppressant, or a combination thereof. In another aspect, a method further comprises administering 5-aminosalicylic acid or a derivative thereof, sulfasalazine or a derivative thereof, or a combination thereof.

In another aspect, a method further comprises administering 5-aminosalicylic acid agent, loperamide, bile acid binders, antidepressants, probiotics, or a combination thereof. In another aspect, a method further comprises administering 5-aminosalicylic acid agent, serotonin synthesis inhibitors, ramosetron, spherical carbon adsorbent, benzodiazepine receptor modulators, peripheral k-agonists, or a combination thereof.

In one aspect, the present disclosure provides a method which eliminates or reduces one or more irritable bowel syndrome symptoms selected from the group consisting of abdominal cramping, abdominal pain, bloating, gas, diarrhea, constipation, hard stool, dry stool, mucus in the stool, depression, anxiety, fatigue, fibromyalgia, sleep disturbances, and chronic headaches.

In another aspect, the present disclosure provides administering anticholinergic drugs, antidiarrheal drugs, or probiotics. In another aspect, the present disclosure provides administering alosetron, rifaximin, eluxadoline, or a combination thereof. In another aspect, the present disclosure provides administering lubiprostone, linaclotide, or a combination thereof. In another aspect, the present disclosure provides administering alosetron, rifaximin, eluxadoline, lubiprostone, linaclotide, or a combination thereof.

In one aspect, the present disclosure provides a method for treating irritable bowel syndrome in a subject in need thereof, where the method comprises administering to the subject a pharmaceutically active dose of a therapeutic composition comprising live non-pathogenic bacteria. In one aspect, the present disclosure provides a method for treating irritable bowel syndrome in a subject in need thereof, where the method comprises administering daily or weekly to the subject a pharmaceutically active dose of a therapeutic composition comprising live non-pathogenic fecal bacteria. In one aspect, a therapeutic composition is administered to an irritable bowel syndrome patient in need thereof at least once daily or at least once weekly for at least two consecutive days or weeks. In one aspect, a therapeutic composition is administered at least once daily or at least once weekly for at least 3, 4, 5, 6, 7, 8, 9, 10, 11, 12, 13, 14, or 15 consecutive days or weeks. In another aspect, a therapeutic composition is administered at least once daily or at least once weekly for at least 1, 2, 3, 4, 5, 6, 7, 8, 9, 10, 11, or 12 consecutive weeks. In one aspect, a therapeutic composition is administered at least once daily or at least once weekly for at most 4, 5, 6, 7, 8, 9, 10, 11, 12, 13, 14, 15, 16, 17, 18, 19, or 20 consecutive days or weeks. In another aspect, a therapeutic composition is administered at least once daily or at least once weekly for at most 1, 2, 3, 4, 5, 6, 7, 8, 9, 10, 11, or 12 consecutive weeks or months. In a further aspect, a therapeutic composition is administered at least once for at least 1, 2, 3, 4, 5, 6, 7, 8, 9, 10, 11, or 12 consecutive months or years, chronically for a subject's entire life span, or an indefinite period of time.

In one aspect, a therapeutic composition is administered to an irritable bowel syndrome patient in need thereof at least twice daily or at least twice weekly for at least two consecutive days or weeks. In one aspect, a therapeutic composition is administered at least twice daily or at least twice weekly for at least 3, 4, 5, 6, 7, 8, 9, 10, 11, 12, 13, 14, or 15 consecutive days or weeks. In another aspect, a therapeutic composition is administered at least twice daily or at least twice weekly for at least 1, 2, 3, 4, 5, 6, 7, 8, 9, 10, 11, or 12 consecutive weeks. In one aspect, a therapeutic composition is administered at least twice daily or at least twice weekly for at most 4, 5, 6, 7, 8, 9, 10, 11, 12, 13, 14, 15, 16, 17, 18, 19, or 20 consecutive days or weeks. In another aspect, a therapeutic composition is administered at least twice daily or at least twice weekly for at most 1, 2, 3, 4, 5, 6, 7, 8, 9, 10, 11, or 12 consecutive weeks or months. In a further aspect, a therapeutic composition is administered at least twice for at least 1, 2, 3, 4, 5, 6, 7, 8, 9, 10, 11, or 12 consecutive months or years, chronically for a subject's entire life span, or an indefinite period of time.

In one aspect, a therapeutic composition is administered to an irritable bowel syndrome patient in need thereof at least three times daily or at least three times weekly for at least two consecutive days or weeks. In one aspect, a therapeutic composition is administered at least three times daily or at least three times weekly for at least 3, 4, 5, 6, 7, 8, 9, 10, 11, 12, 13, 14, or 15 consecutive days or weeks. In another aspect, a therapeutic composition is administered at least three times daily or at least three times weekly for at least 1, 2, 3, 4, 5, 6, 7, 8, 9, 10, 11, or 12 consecutive weeks. In one aspect, a therapeutic composition is administered at least three times daily or at least three times weekly for at most 4, 5, 6, 7, 8, 9, 10, 11, 12, 13, 14, 15, 16, 17, 18, 19, or 20 consecutive days or weeks. In another aspect, a therapeutic composition is administered at least three times daily or at least three times weekly for at most 1, 2, 3, 4, 5, 6, 7, 8, 9, 10, 11, or 12 consecutive weeks or months. In a further aspect, a therapeutic composition is administered at least three times for at least 1, 2, 3, 4, 5, 6, 7, 8, 9, 10, 11, or 12 consecutive months or years, chronically for a subject's entire life span, or an indefinite period of time.

In one aspect, the present disclosure provides a method for treating irritable bowel syndrome in a subject in need thereof, where the method comprises administering orally to the subject a pharmaceutically active dose of a therapeutic composition comprising live, non-pathogenic, synthetic bacterial mixture or live, non-pathogenic, purified or extracted, fecal microbiota, where the dose is administered at a dosing schedule of at least once or twice daily or at least once or twice weekly for at least three consecutive days or weeks. In another aspect, a dose is administered at least once, twice, or three times daily or at least once, twice, or three times weekly for a period between 1 and 12 weeks, between 2 and 12 weeks, between 3 and 12 weeks, between 4 and 12 weeks, between 5 and 12 weeks, between 6 and 12 weeks, between 7 and 12 weeks, between 8 and 12 weeks, between 9 and 12 weeks, between 10 and 12 weeks, between 1 and 2 weeks, between 2 and 3 weeks, between 3 and 4 weeks, between 4 and 5 weeks, between 5 and 6 weeks, between 6 and 7 weeks, between 7 and 8 weeks, between 8 and 9 weeks, between 9 and 10 weeks, or between 10 and 11 weeks.

In one aspect, the present disclosure provides a method for treating irritable bowel syndrome in a subject in need thereof, where the method comprises a first dosing schedule followed by a second dosing schedule. In one aspect, a first dosing schedule comprises a treatment or induction dose. In one aspect, a first dosing schedule comprises a continuous dosing schedule. In another aspect, a second dosing schedule comprises a maintenance dose lower than or equal to a pharmaceutically active dose of a first dosing schedule. In another aspect, a second dosing schedule lasts for at least about 2, 4, 6, 8, 10, 12, 18, 24, 36, 48, 72, or 96 months. In one aspect, a second dosing schedule lasts permanently, for a treated subject's entire life span, or an indefinite period of time. In one aspect, a second dosing schedule is a continuous dosing schedule. In another aspect, a second dosing schedule is an intermittent dosing schedule. In a further aspect, a second dosing schedule is an intermittent dosing schedule comprising a treatment period of at least 1, 2, 3, 4, 5, 6, 7, 8, 9, 10, 11, 12, 13, or 14 days followed by a resting period of at least 1, 2, 3, 4, 5, 6, 7, 8, 9, 10, 11, 12, 13, or 14 days. In another aspect, a second dosing schedule comprises administering a second dose (e.g., a maintenance dose) every other day, every two days, or every 3, 4, 5, 6, 7, 8 days. In another aspect, a maintenance dose is administered for an extended period of time with or without titration (or otherwise changing the dosage or dosing schedule). In one aspect, the interval between a first and a second dosing schedule is at least about 1, 2, 3, 4, 5, 6, 7, 8, 9, 10, 11, or 12 weeks. In another aspect, a second dosing schedule (e.g., a maintenance dose) comprises a dosage about 2, 5, 10, 50, 100, 200, 400, 800, 1000, 5000 or more folds lower than the dosage used in a first dosing schedule (e.g., an initial treatment dose). In another aspect, a second dosing schedule (e.g., a maintenance dosing schedule) has an equal or lower dosing frequency than a first dosing schedule (e.g., an initial treatment dosing schedule). In another aspect, a second dosing schedule (e.g., a maintenance dosing schedule) has a higher dosing interval than a first dosing schedule (e.g., an initial treatment dosing schedule).

In one aspect, a first or second dosing schedule used in a method can be once-a-week, twice-a-week, or thrice-a-week. The term “once-a-week” means that a dose is administered once in a week, preferably on the same day of each week. “Twice-a-week” means that a dose is administered two times in a week, preferably on the same two days of each weekly period. “Thrice-a-week” means that a dose is administered three times in a week, preferably on the same three days of each weekly period.

In one aspect, a subject being treated is a subject already with irritable bowel syndrome. Administration of a disclosed therapeutic composition to clinically, asymptomatic human subject who is genetically predisposed or prone to irritable bowel syndrome is also useful in preventing the onset of clinical symptoms of irritable bowel syndrome. A human subject genetically predisposed or prone to irritable bowel syndrome can be a human subject having a close family member or relative exhibiting or having suffered irritable bowel syndrome. In another aspect, a subject being treated is a subject in which irritable bowel syndrome is to be prevented. In another aspect, a subject being treated is predisposed or susceptible to irritable bowel syndrome. In another aspect, a subject being treated is a subject diagnosed as having irritable bowel syndrome. In one aspect, a subject being treated is a patient in need thereof. In another aspect, a patient being treated is immunocompromised.

In one aspect, a subject being treated is a human patient. In one aspect, a patient is a male patient. In one aspect, a patient is a female patient. In one aspect, a patient is a premuature newborn. In one aspect, a patient is a term newborn. In one aspect, a patient is a neonate. In one aspect, a patient is an infant. In one aspect, a patient is a toddler. In one aspect, a patient is a young child. In one aspect, a patient is a child. In one aspect, a patient is an adolescent. In one aspect, a patient is a pediatric patient. In one aspect, a patient is a geriatric patient. In one aspect, a human patient is a child patient below about 18, 15, 12, 10, 8, 6, 4, 3, 2, or 1 year old. In another aspect, a human patient is an adult patient. In another aspect, a human patient is an elderly patient. In a further aspect, a human patient is a patient above about 30, 35, 40, 45, 50, 55, 60, 65, 70, 75, 80, 85, 90, or 95 years old. In another aspect, a patient is about between 1 and 5, between 2 and 10, between 3 and 18, between 21 and 50, between 21 and 40, between 21 and 30, between 50 and 90, between 60 and 90, between 70 and 90, between 60 and 80, or between 65 and 75 years old. In one aspect, a patient is a young old patient (65-74 years). In one aspect, a patient is a middle old patient (75-84 years). In one aspect, a patient is an old old patient (>85 years).

In one aspect, a method comprises administering a therapeutic composition orally, by enema, or via rectal suppository. In one aspect, a therapeutic composition administered herein is formulated as an enteric coated (and/or acid-resistant) capsule or microcapsule, or formulated as part of or administered together with a food, a food additive, a dairy-based product, a soy-based product or a derivative thereof, a jelly, flavored liquid, ice block, ice-cream, or a yogurt. In another aspect, a therapeutic composition administered herein is formulated as an acid-resistant enteric coated capsule. A therapeutic composition can be provided as a powder for sale in combination with a food or drink. A food or drink can be a dairy-based product or a soy-based product. In another aspect, a food or food supplement contains enteric-coated and/or acid-resistant microcapsules containing a therapeutic composition.

In an aspect, a therapeutic composition comprises a liquid culture. In another aspect, a therapeutic composition is lyophilized, pulverized and powdered. It may then be infused, dissolved such as in saline, as an enema. Alternatively the powder may be encapsulated as enteric-coated and/or acid-resistant capsules for oral administration. These capsules may take the form of enteric-coated and/or acid-resistant microcapsules. A powder can preferably be provided in a palatable form for reconstitution for drinking or for reconstitution as a food additive. In a further aspect, a food is yogurt. In one aspect, a powder may be reconstituted to be infused via naso-duodenal infusion.

In another aspect, a therapeutic composition administered herein is in a liquid, frozen, freeze-dried, spray-dried, foam-dried, lyophilized, or powder form. In a further aspect, a therapeutic composition administered herein is formulated as a delayed or gradual enteric release form. In another aspect, a therapeutic composition administered herein comprises an excipient, a saline, a buffer, a buffering agent, or a fluid-glucose-cellobiose agar (RGCA) media. In another aspect, a therapeutic composition administered herein comprises a cryoprotectant. In one aspect, a cryoprotectant comprises polyethylene glycol, skim milk, erythritol, arabitol, sorbitol, glucose, fructose, alanine, glycine, proline, sucrose, lactose, ribose, trehalose, dimethyl sulfoxide (DMSO), glycerol, or a combination thereof.

In one aspect, a therapeutic composition administered herein further comprises an acid suppressant, an antacid, an H2 antagonist, a proton pump inhibitor or a combination thereof. In one aspect, a therapeutic composition administered herein substantially free of non-living matter. In another aspect, a therapeutic composition administered herein substantially free of acellular material selected from the group consisting of residual fiber, DNA, viral coat material, and non-viable material.

In one aspect, a therapeutic composition also comprises or is supplemented with a prebiotic nutrient selected from the group consisting of polyols, fructooligosaccharides (FOSs), oligofructoses, inulins, galactooligosaccharides (GOSs), xylooligosaccharides (XOSs), polydextroses, monosaccharides, tagatose, and/or mannooligosaccharides.

In one aspect, a method further comprises pretreating a subject with an antibiotic composition prior to administering a therapeutic bacterial or microbiota composition. In one aspect, an antibiotic composition administered herein comprises an antibiotic selected from the group consisting of rifabutin, clarithromycin, clofazimine, vancomycin, rifampicin, nitroimidazole, chloramphenicol, metronidazole, colchicine and a combination thereof. In another aspect, an antibiotic composition administered herein comprises an antibiotic selected from the group consisting of rifaximin, rifamycin derivative, rifampicin, rifabutin, rifapentine, rifalazil, bicozamycin, aminoglycoside, gentamycin, neomycin, streptomycin, paromomycin, verdamicin, mutamicin, sisomicin, netilmicin, retymicin, kanamycin, aztreonam, aztreonam macrolide, clarithromycin, dirithromycin, roxithromycin, telithromycin, azithromycin, bismuth subsalicylate, vancomycin, streptomycin, fidaxomicin, amikacin, arbekacin, neomycin, netilmicin, paromomycin, rhodostreptomycin, tobramycin, apramycin, metronidazole, colchicine, and a combination thereof. In a further aspect, a method further comprises pretreating a subject with an anti-inflammatory drug prior to administration of a therapeutic bacterial or microbiota composition.

In one aspect, a method achieves a remission, cure, response, or resolution rate of irritable bowel syndrome of at least about 10%, 15%, 20%, 25%, 30%, 35%, 40%, 45%, 50%, 55%, 60%, 65%, 70%, 75%, 80%, 85%, 90%, 95%, 97%, or 99%. In another aspect, a method achieves a remission, cure, response, or resolution rate of irritable bowel syndrome of between about 10-20%, 20-30%, 30-40%, 40-50%, 50-60%, 60-70%, 70-80%, 80-90%, or 90-99%. In a further aspect, a patient is assessed using the Subject's Global Assessment of Relief (SGA) as described in Muller-Lissner et al., Subject's global assessment of relief: an appropriate method to assess the impact of treatment on irritable bowel syndrome-related symptoms in clinical trials. Journal of Clin. Epidemiology. 2003; 56(4): 310-316 and FDA Center for Drugs Evaluation and Research. Guidance for Industry Irritable Bowel Syndrome-clinical evaluation of drugs for treatment. Clinical/Medical 2012. In one aspect, a treatment method achieves a reduction of irritable bowel syndrome after 4, 8, or 12 weeks of treatment in at least 10%, 20%, 30%, 40%, 50%, 60%, 70%, 80%, or 90% patients in a patient population. In another aspect, a treatment method achieves a reduction of irritable bowel syndrome after 4, 8, or 12 weeks of treatment in between about 10-20%, 20-30%, 30-40%, 40-50%, 50-60%, 60-70%, 70-80%, 80-90%, or 90-99% patients in a patient population. In one aspect, a treatment method achieves at least 10%, 20%, 30%, 50%, 60%, 70%, 80%, or 90% reduction of irritable bowel syndrome after 4, 8, or 12 weeks of treatment compared to baseline (e.g., immediately prior to treatment). In another aspect, a treatment method achieves between about 10-20%, 20-30%, 30-40%, 40-50%, 50-60%, 60-70%, 70-80%, 80-90%, or 90-99% reduction of irritable bowel syndrome after 4, 8, or 12 weeks of treatment compared to baseline (e.g., immediately prior to treatment). In one aspect, a treatment method achieves at least 10%, 20%, 30%, 50%, 60%, 70%, 80%, or 90% reduction of irritable bowel syndrome in at least 10%, 20%, 30%, 50%, 60%, 70%, 80%, or 90% patients after 4, 8, or 12 weeks of treatment compared to baseline (e.g., immediately prior to treatment). In a further aspect, a treatment method achieves between about 10-20%, 20-30%, 30-40%, 40-50%, 50-60%, 60-70%, 70-80%, 80-90%, or 90-99% reduction of irritable bowel syndrome in between about 10-20%, 20-30%, 30-40%, 40-50%, 50-60%, 60-70%, 70-80%, 80-90%, or 90-99% patients after 4, 8, or 12 weeks of treatment compared to baseline (e.g., immediately prior to treatment).

In a further aspect, a patient is assessed using the irritable bowel syndrome symptom severity scoring system (MS SSS) as described in Francis et al., The irritable bowel severity scoring system: a simple method of monitoring irritable bowel syndrome and its progress. Aliment Parmacol Ther. 1997; 11:395-402. In one aspect, a treatment method achieves at least 10%, 20%, 30%, 50%, 60%, 70%, 80%, or 90% reduction of irritable bowel syndrome symptom severity score (MS SSS) after 4, 8, or 12 weeks of treatment compared to baseline (e.g., immediately prior to treatment). In another aspect, a treatment method achieves at least 10-20%, 20-30%, 30-40%, 40-50%, 50-60%, 60-70%, 70-80%, 80-90 or 90-99% reduction of irritable bowel syndrome symptom severity score (MS SSS) after 4, 8, or 12 weeks of treatment compared to baseline (e.g., immediately prior to treatment). In one aspect, a treatment method achieves a reduction of irritable bowel syndrome symptom severity score (MS SSS) after 4, 8, or 12 weeks of treatment by more than 2, 3, 4, 5, 6, 7, 8, 9, 10, or 11. In another aspect, a treatment method achieves a reduction of irritable bowel syndrome symptom severity score (MS SSS) after 4, 8, or 12 weeks of treatment by more than 2-4, 4-6, 6-8, 8-10, or 10-11. In one aspect, a treatment method achieves a reduction of irritable bowel syndrome symptom severity score (MS SSS) after 4, 8, or 12 weeks of treatment by more than 10, 20, 30, 40, 50, 60, 80, 90, or 100. In yet another aspect, a treatment method achieves a reduction of irritable bowel syndrome symptom severity score (MS SSS) after 4, 8, or 12 weeks of treatment by between about 10-20, 20-30, 30-40, 40-50, 50-60, 60-70, 70-80, 80-90, or 90-100. In one aspect, a treatment method achieves a reduction of irritable bowel syndrome symptom severity score (MS SSS) after 4, 8, or 12 weeks of treatment by more than 100, 200, 300, or 425. In another aspect, a treatment method achieves a reduction of irritable bowel syndrome symptom severity score (MS SSS) after 4, 8, or 12 weeks of treatment by between about 100-150, 150-200, 200-300, or 300-425. In one aspect, a treatment method achieves a reduction of irritable bowel syndrome symptom severity score (MS SSS) after 4, 8, or 12 weeks of treatment by between 70-500. In another aspect, a treatment method achieves a reduction of irritable bowel syndrome symptom severity scoring system (MS SSS) after 4, 8, or 12 weeks of treatment by between 75-425. In a further aspect, a treatment method achieves a reduction of irritable bowel syndrome symptom severity scoring system (MS SSS) after 4, 8, or 12 weeks of treatment by between about 150-425. In another aspect, a treatment method achieves a reduction of irritable bowel syndrome symptom severity score (MS SSS) after 4, 8, or 12 weeks of treatment by between 70 to 500 in at least 10%, 20%, 30%, 50%, 60%, 70%, 80%, or 90% patients in a patient population. In a further aspect, a treatment method achieves a reduction of irritable bowel syndrome symptom severity score (MS SSS) after 4, 8, or 12 weeks of treatment by between about 70-500 in between about 10-20%, 20-30%, 30-40%, 40-50%, 50-60%, 60-70%, 70-80%, 80-90%, or 90-100% patients in a patient population. In one aspect, a treatment method achieves at least 10%, 20%, 30%, 50%, 60%, 70%, 80%, or 90% reduction of IBS symptom severity score in at least 10%, 20%, 30%, 50%, 60%, 70%, 80%, or 90% patients after 4, 8, or 12 weeks of treatment compared to baseline (e.g., immediately prior to treatment). In another aspect, a treatment method achieves in between about 10-20%, 20-30%, 30-40%, 40-50%, 50-60%, 60-70%, 70-80%, 80-90%, or 90-100% reduction of IBS symptom severity score in between about 10-20%, 20-30%, 30-40%, 40-50%, 50-60%, 60-70%, 70-80%, 80-90%, or 90-100% patients after 4, 8, or 12 weeks of treatment compared to baseline (e.g., immediately prior to treatment).

In one aspect, every about 200 mg of a pharmaceutical composition comprises a pharmacologically active dose. In one aspect, every about 75, 100, 125, 150, 175, 200, 250, 300, 350, 400, 450, 500, 750, 1000, 1500, or 2000 mg of a pharmaceutical composition comprises a pharmacologically active dose.

In one aspect, a pharmaceutically active or therapeutic effective dose comprises at least about 10⁵, 10⁶, 10⁷, 10⁸, 10⁹, 10¹⁰, 10¹¹, 10¹², 10¹³, 10¹⁴, or 10¹⁵ cfu. In another aspect, a pharmaceutically active therapeutic effective dose comprises at most about 10⁵, 10⁶, 10⁷, 10⁸, 10⁹, 10¹⁰, 10¹¹, 10¹², or 10¹³, 10¹⁴, or 10¹⁵ cfu. In a further aspect, a pharmacologically active therapeutic effective dose is selected from the group consisting of from 10⁸ cfu to 10¹⁴ cfu, from 10⁹ cfu to 10¹³ cfu, from 10¹⁰ cfu to 10¹² cfu, from 10⁹ cfu to 10¹⁴ cfu, from 10⁹ cfu to 10¹² cfu, from 10⁹ cfu to 10¹¹ cfu, from 10⁹ cfu to 10¹⁰ cfu, from 10¹⁰ cfu to 10¹⁴ cfu, from 10¹⁰ cfu to 10¹³ cfu, from 10¹¹ cfu to 10¹⁴ cfu, from 10¹¹ cfu to 10¹³ cfu, from 10¹² cfu to 10¹⁴ cfu, and from 10¹³ cfu to 10¹⁴ cfu. In one aspect, a pharmaceutical composition comprises the foregoing pharmaceutically active or therapeutic effective dose in a unit weight of about 0.2, 0.4, 0.6, 0.8 or 1.0 gram, or a unit volume of about 0.2, 0.4, 0.6, 0.8 or 1.0 milliliter.

In one aspect, a pharmaceutically active or therapeutic effective dose comprises at least about 10⁵, 10⁶, 10⁷, 10⁸, 10⁹, 10¹⁰, 10¹¹, 10¹², 10¹³, 10¹⁴, or 10¹⁵ cells or spores. In another aspect, a pharmaceutically active or therapeutic effective dose comprises at most about 10⁵, 10⁶, 10⁷, 10⁸, 10⁹, 10¹⁰, 10¹¹, 10¹², 10¹³, 10¹⁴, or 10¹⁵ total cells or spores. In a further aspect, a pharmacologically active or therapeutic effective dose is selected from the group consisting of from 10⁸ to 10¹⁴, from 10⁹ to 10¹³, from 10¹⁰ to 10¹², from 10⁹ to 10¹⁴, from 10⁹ to 10¹², from 10⁹ to 10¹¹, from 10⁹ to 10¹⁰, from 10¹⁰ to 10¹⁴, from 10¹⁰ to 10¹³, from 10¹¹ to 10¹⁴, from 10¹¹ to 10¹³, from 10¹² to 10¹⁴, and from 10¹³ to 10¹⁴ cells or spores. In an aspect, the pharmaceutically active or therapeutic effective dose cell count is directed to live cells. In one aspect, a pharmaceutical composition comprises the foregoing pharmaceutically active or therapeutic effective dose in a unit weight of about 0.2, 0.4, 0.6, 0.8 or 1.0 gram, or a unit volume of about 0.2, 0.4, 0.6, 0.8 or 1.0 milliliter. In an aspect, a pharmaceutically active or therapeutic effective dose comprises between 10¹⁰ and 10¹² cells. In another aspect, a pharmaceutically active or therapeutic effective dose comprises between 10¹⁰ and 10¹² cells per capsule.

In one aspect, a therapeutic composition administered herein comprises fecal bacteria. In one aspect, a therapeutic composition administered herein comprises one or more, two or more, three or more, four or more, or five or more isolated, purified, or cultured microorganisms selected from the group consisting of Clostridium, Bacillus, Collinsella, Bacteroides, Eubacterium, Fusobacterium, Propionibacterium, Lactobacillus, Ruminococcus, Escherichia coli, Gemmiger, Desulfomonas, Peptostreptococcus, Bifidobacterium, Coprococcus, Dorea, and Monilia.

In one aspect, a therapeutic composition administered herein comprises at least one, at least two, at least three, at least four, at least five, at least six, or at least seven fecal microorganisms selected from the group consisting of a Bacteroides fragilis ssp. vulgatus, Collinsella aerofaciens, Bacteroides fragilis ssp. thetaiotaomicron, Peptostreptococcus productus II, Parabacteroides distasonis, Fusobacterium prausnitzii, Coprococcus eutactus, Collinsella aerofaciens III, Peptostreptococcus productus I, Ruminococcus bromii, Bifidobacterium adolescentis, Gemmiger formicilis, Bifidobacterium longum, Eubacterium siraeum, Ruminococcus torques, Eubacterium rectale, Eubacterium eligens, Bacteroides eggerthii, Clostridium leptum, Bacteroides fragilis ssp. A, Eubacterium biforme, Bifidobacterium infantis, Eubacterium rectale Coprococcus comes, Pseudoflavonifractor capillosus, Ruminococcus albus, Dorea formicigenerans, Eubacterium hallii, Eubacterium ventriosum I, Fusobacterium russi, Ruminococcus obeum, Eubacterium rectale, Clostridium ramosum, Lactobacillus leichmannii, Ruminococcus callidus, Butyrivibrio crossotus, Acidaminococcus fermentans, Eubacterium ventriosum, Bacteroides fragilis ssp. fragilis, Bacteroides AR, Coprococcus catus, Aerostipes hadrus, Eubacterium cylindroides, Eubacterium ruminantium, Eubacterium CH-1, Staphylococcus epidermidis, Peptostreptococcus BL, Eubacterium limosum, Tissirella praeacuta, Bacteroides L, Fusobacterium mortiferum I, Fusobacterium naviforme, Clostridium innocuum, Clostridium ramosum, Propionibacterium acnes, Ruminococcus flavefaciens, Ruminococcus AT, Peptococcus AU-1, Bacteroides fragilis ssp. ovatus, -ssp. d, -ssp. f; Bacteroides L-1, L-5; Fusobacterium nucleatum, Fusobacterium mortiferum, Escherichia coli, Gemella morbillorum, Finegoldia magnus, Peptococcus G, -AU-2; Streptococcus intermedius, Ruminococcus lactaris, Ruminococcus CO Gemmiger X, Coprococcus BH, —CC; Eubacterium tenue, Eubacterium ramulus, Bacteroides clostridiiformis ssp. clostridiiformis, Bacteroides coagulans, Prevotella oxalis, Prevotella ruminicola, Odoribacter splanchnicus, Desuifomonas pigra, Lactobacillus G, Succinivibrio A, and a combination thereof.

In one aspect, a therapeutic composition administered herein comprises no viable Bacteroides, Fusobacterium, Propionibacterium, Lactobacillus, Ruminococcus, Escherichia coli, Gemmiger, Desulfomonas, Peptostreptococcus, Bifidobacterium, Monilia, or any combination thereof. In another aspect, a therapeutic composition administered herein comprises no viable Bacteroides fragilis ssp. vulgatus, Collinsella aerofaciens, Bacteroides fragilis ssp. thetaiotaomicron, Peptostreptococcus productus II, Parabacteroides distasonis, Fusobacterium prausnitzii, Coprococcus eutactus, Collinsella aerofaciens III, Peptostreptococcus productus I, Ruminococcus bromii, Bifidobacterium adolescentis, Gemmiger formicilis, Bifidobacterium longum, Eubacterium siraeum, Ruminococcus torques, Eubacterium rectale, Eubacterium eligens, Bacteroides eggerthii, Clostridium leptum, Bacteroides fragilis ssp. A, Eubacterium biforme, Bifidobacterium infantis, Eubacterium rectale III-F, Coprococcus comes, Pseudoflavonifractor capillosus, Ruminococcus albus, Dorea formicigenerans, Eubacterium hallii, Eubacterium ventriosum I, Fusobacterium russi, Ruminococcus obeum, Eubacterium rectale, Clostridium ramosum, Lactobacillus leichmannii, Ruminococcus callidus, Butyrivibrio crossotus, Acidaminococcus fermentans, Eubacterium ventriosum, Bacteroides fragilis ssp. fragilis, Bacteroides AR, Coprococcus catus, Aerostipes hadrus, Eubacterium cylindroides, Eubacterium ruminantium, Eubacterium CH-1, Staphylococcus epidermidis, Peptostreptococcus BL, Eubacterium limosum, Tissirella praeacuta, Bacteroides L, Fusobacterium mortiferum I, Fusobacterium naviforme, Clostridium innocuum, Clostridium ramosum, Propionibacterium acnes, Ruminococcus flavefaciens, Ruminococcus AT, Peptococcus AU-1, Bacteroides fragilis ssp. ovatus, -ssp. d, -ssp. f; Bacteroides L-1, L-5; Fusobacterium nucleatum, Fusobacterium mortiferum, Escherichia coli, Gemella morbillorum, Finegoldia magnus, Peptococcus G, -AU-2; Streptococcus intermedius, Ruminococcus lactaris, Ruminococcus CO Gemmiger X, Coprococcus BH, —CC; Eubacterium tenue, Eubacterium ramulus, Bacteroides clostridiiformis ssp. clostridliformis, Bacteroides coagulans, Prevotella oralis, Prevotella ruminicola, Odoribacter splanchnicus, Desuifomonas pigra, Lactobacillus G, Succinivibrio A, or a combination thereof.

In one aspect, a therapeutic composition administered herein comprises a fecal microbiota. In another aspect, the preparation of a fecal microbiota used herein involves a treatment selected from the group consisting of ethanol treatment, detergent treatment, heat treatment, irradiation, and sonication. In another aspect, the preparation of a fecal microbiota used herein involves no treatment selected from the group consisting of ethanol treatment, detergent treatment, heat treatment, irradiation, and sonication. In one aspect, the preparation of a fecal microbiota used herein involves a separation step selected from the group consisting of density gradients, filtration (e.g., sieves, nylon mesh), and chromatography. In another aspect, the preparation of a fecal microbiota used herein involves no separation step selected from the group consisting of density gradients, filtration (e.g., sieves, nylon mesh), and chromatography. In another aspect, a fecal microbiota used herein comprises a donor's entire fecal microbiota. In another aspect, a therapeutic composition administered herein comprises a fecal microbiota substantially free of eukaryotic cells from the fecal microbiota's donor.

In another aspect, a therapeutic composition administered herein comprises a fecal microbiota further supplemented, spiked, or enhanced with a fecal microorganism. In one aspect, a fecal microbiota is supplemented with a non-pathogenic (or with attenuated pathogenicity) bacterium of Clostridium, Collinsella, Dorea, Ruminococcus, Coprococcus, Prevotella, Veillonella, Bacteroides, Baccillus, or a combination thereof. In another aspect, a therapeutic composition administered herein comprises a fecal microbiota further supplemented, spiked, or enhanced with a species of Veillonellaceae, Firmicutes, Gammaproteobacteria, Bacteroidetes, or a combination thereof. In another aspect, a therapeutic composition administered herein comprises a fecal microbiota further supplemented with fecal bacterial spores. In one aspect, fecal bacterial spores are Clostridium spores, Bacillus spores, or both.

In an aspect, a therapeutic composition comprises a fecal microbiota from a subject selected from the group consisting of a human, a bovine, a dairy calf, a ruminant, an ovine, a caprine, or a cervine. In another aspect, a therapeutic composition can be administered to a subject selected from the group consisting of a human, a bovine, a dairy calf, a ruminant, an ovine, a caprine, or a cervine. In an aspect, a therapeutic composition is substantially or nearly odourless.

In an aspect, a therapeutic composition provided or administered herein comprises a fecal microbiota comprising a Shannon Diversity Index of greater than or equal to 0.3, greater than or equal to 0.4, greater than or equal to 0.5, greater than or equal to 0.6, greater than or equal to 0.7, greater than or equal to 0.8, greater than or equal to 0.9, greater than or equal to 1.0, greater than or equal to 1.1, greater than or equal to 1.2, greater than or equal to 1.3, greater than or equal to 1.4, greater than or equal to 1.5, greater than or equal to 1.6, greater than or equal to 1.7, greater than or equal to 1.8, greater than or equal to 1.9, greater than or equal to 2.0, greater than or equal to 2.1, greater than or equal to 2.2, greater than or equal to 2.3, greater than or equal to 2.4, greater than or equal to 2.5, greater than or equal to 3.0, greater than or equal to 3.1, greater than or equal to 3.2, greater than or equal to 3.3, greater than or equal to 3.4, greater than or equal to 3.5, greater than or equal to 3.6, greater than or equal to 3.7, greater than or equal to 3.8, greater than or equal to 3.9, greater than or equal to 4.0, greater than or equal to 4.1, greater than or equal to 4.2, greater than or equal to 4.3, greater than or equal to 4.4, greater than or equal to 4.5, or greater than or equal to 5.0. In another aspect, a therapeutic composition comprises fecal microbiota comprising a Shannon Diversity Index of between 0.1 and 3.0, between 0.1 and 2.5, between 0.1 and 2.4, between 0.1 and 2.3, between 0.1 and 2.2, between 0.1 and 2.1, between 0.1 and 2.0, between 0.4 and 2.5, between 0.4 and 3.0, between 0.5 and 5.0, between 0.7 and 5.0, between 0.9 and 5.0, between 1.1 and 5.0, between 1.3 and 5.0, between 1.5 and 5.0, between 1.7 and 5.0, between 1.9 and 5.0, between 2.1 and 5.0, between 2.3 and 5.0, between 2.5 and 5.0, between 2.7 and 5.0, between 2.9 and 5.0, between 3.1 and 5.0, between 3.3 and 5.0, between 3.5 and 5.0, between 3.7 and 5.0, between 31.9 and 5.0, or between 4.1 and 5.0. In one aspect, a Shannon Diversity Index is calculated at the phylum level. In another aspect, a Shannon Diversity Index is calculated at the family level. In one aspect, a Shannon Diversity Index is calculated at the genus level. In another aspect, a Shannon Diversity Index is calculated at the species level. In a further aspect, a therapeutic composition comprises a preparation of flora in proportional content that resembles a normal healthy human fecal flora.

In a further aspect, a therapeutic composition comprises fecal bacteria from at least 1, 2, 3, 4, 5, 6, 7, 8, 9, or 10 different families. In another aspect, a therapeutic composition comprises fecal bacteria from at least 11, 12, 13, 14, 15, 16, 17, 18, 19, or 20 different families. In yet another aspect, a therapeutic composition comprises fecal bacteria from at least 21, 22, 23, 24, 25, 26, 27, 28, 29, or 30 different families. In a further aspect, a therapeutic composition comprises fecal bacteria from at least 31, 32, 33, 34, 35, 36, 37, 38, 39, or 40 different families. In another aspect, a therapeutic composition comprises fecal bacteria from at least 41, 42, 43, 44, 45, 46, 47, 48, 49, or 50 different families. In another aspect, a therapeutic composition comprises fecal bacteria from between 1 and 10, between 10 and 20, between 20 and 30, between 30 and 40, between 40 and 50 different families. In an aspect, a therapeutic composition provided or administered herein comprises a fecal microbiota comprising no greater than 0.05%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, or 10% weight non-living material/weight biological material. In another aspect, a therapeutic composition provided or administered herein comprises a fecal microbiota comprising no greater than 20%, 25%, 30%, 35%, 40%, 45%, 50%, 55%, 60%, 65%, 70%, 75%, 80%, 85%, 90%, or 95% weight non-living material/weight biological material. In another aspect, a therapeutic composition provided or administered herein comprises, consists of, or consists essentially of, particles of non-living material and/or particles of biological material of a fecal sample that passes through a sieve, a column, or a similar filtering device having a sieve, exclusion, or particle filter size of 2.0 mm, 1.0 mm, 0.5 mm, 0.33 mm, 0.25 mm, 0.212 mm, 0.180 mm, 0.150 mm, 0.125 mm, 0.106 mm, 0.090 mm, 0.075 mm, 0.063 mm, 0.053 mm, 0.045 mm, 0.038 mm, 0.032 mm, 0.025 mm, 0.020 mm, 0.01 mm, 0.002 mm, 0.1 mm, or 0.2 mm. “Non-living material” does not include an excipient, e.g., a pharmaceutically inactive substance, such as a cryoprotectant, added to a processed fecal material. “Biological material” refers to the living material in fecal material, and includes microbes including prokaryotic cells, such as bacteria and archaea (e.g., living prokaryotic cells and spores that can sporulate to become living prokaryotic cells), eukaryotic cells such as protozoa and fungi, and viruses. In one embodiment, “biological material” refers to the living material, e.g., the microbes, eukaryotic cells, and viruses, which are present in the colon of a normal healthy human. In an aspect, a therapeutic composition provided or administered herein comprises an extract of human feces where the composition is substantially odorless. In an aspect, a therapeutic composition provided or administered herein comprises fecal material or a fecal floral preparation in a lyophilized, crude, semi-purified or purified formulation.

In an aspect, a fecal microbiota in a therapeutic composition comprises highly refined or purified fecal flora, e.g., substantially free of non-floral fecal material. In an aspect, a fecal microbiota can be further processed, e.g., to undergo microfiltration before, after, or before and after sieving. In another aspect, a highly purified fecal microbiota product is ultra-filtrated to remove large molecules but retain the therapeutic microflora, e.g., bacteria.

In another aspect, a fecal microbiota in a therapeutic composition used herein comprises or consists essentially of a substantially isolated or a purified fecal flora or entire (or substantially entire) microbiota that is (or comprises) an isolate of fecal flora that is at least about 90%, 91%, 92%, 93%, 94%, 95%, 96%, 97%, 98%, 99%, 99.5%, 99.6%, 99.7%, 99.8% or 99.9% isolated or pure, or having no more than about 0.1%, 0.2%, 0.3%, 0.4%, 0.5%, 0.6%, 0.7%, 0.8%, 0.9% or 1.0% or more non-fecal floral material; or, a substantially isolated, purified, or substantially entire microbiota as described in Sadowsky et al., WO 2012/122478 A1, or as described in Borody et al., WO 2012/016287 A2.

In an aspect, a fecal microbiota in a therapeutic composition comprises a donor's substantially entire or non-selected fecal microbiota, reconstituted fecal material, or synthetic fecal material. In another aspect, the fecal microbiota in a therapeutic composition comprises no antibiotic resistant population. In another aspect, a therapeutic composition comprises a fecal microbiota and is largely free of extraneous matter (e.g., non-living matter including acellular matter such as residual fiber, DNA, RNA, viral coat material, non-viable material; and living matter such as eukaryotic cells from the fecal matter's donor).

In an aspect, a fecal microbiota in a therapeutic composition used herein is derived from disease-screened fresh homologous feces or equivalent freeze-dried and reconstituted feces. In an aspect, a fresh homologous feces does not include an antibiotic resistant population. In another aspect, a fecal microbiota in a therapeutic composition is derived from a synthetic fecal composition. In an aspect, a synthetic fecal composition comprises a preparation of viable flora which preferably in proportional content, resembles normal healthy human fecal flora which does not include antibiotic resistant populations. Suitable microorganisms may be selected from the following: Bacteroides, Eubacterium, Fusobacterium, Propionibacterium, Lactobacillus, Ruminococcus, Escherichia coli, Gemmiger, Clostridium, Desulfomonas, Peptostreptococcus, Bifidobacterium, Collinsella, Coprococcus, Dorea, and Ruminococcus.

In an aspect, a therapeutic composition used in a treatment disclosed herein comprises a sterile fecal filtrate or a non-cellular fecal filtrate. In one aspect, a sterile fecal filtrate originates from a donor stool. In another aspect, a sterile fecal filtrate originates from cultured microorganisms. In another aspect, a sterile fecal filtrate comprises a non-cellular non-particulate fecal component. In one aspect, a sterile fecal filtrate is made as described in WO2014/078911, published May 30, 2014. In another aspect, a sterile fecal filtrate is made as described in Ott et al., Gastroenterology 152:799-911(2017).

In one aspect, a fecal filtrate comprises secreted, execreted or otherwise liquid components or a microbiota, e.g., biologically active molecules (BAMs), which can be antibiotics or anti- inflammatories, are preserved, retained or reconstituted in a flora extract.

In one aspect, an exemplary therapeutic composition comprises starting material from a donor from a defined donor pool, where this donor contributes a stool that is centrifuged, then filtered with very high-level filtration using e.g., either metal sieving or Millipore filters, or equivalent, to ultimately permit only cells of bacterial origin to remain, e.g., often less than about 5 micrometers diameter. After the initial centrifugation, the solid material is separated from the liquid, and the solid is then filtered in progressively reducing size filters and tangential filters, e.g., using a Millipore filtration, and optionally, also comprising use of nano-membrane filtering. The filtering can also be done by sieves as described in WO 2012/122478, but in contrast using sieves that are smaller than 0.0120 mm, down to about 0.0110 mm, which ultimately result in having only bacterial cells present.

The supernatant separated during centrifugation is now taken and filtered progressively in a filtering, e.g., a Millipore filtering or equivalent systems, to end up with liquid which is finely filtered through an about 0.22 micron filter. This removes all particulate matter including all living matter, including bacteria and viruses. The product then is sterile, but the aim is to remove the bacteria but to keep their secretions, especially antimicrobial bacteriocins, bacteria-derived cytokine-like products and all accompanying Biologically Active Molecules (BAMs), including: thuricin (which is secreted by bacilli in donor stools), bacteriocins (including colicin, troudulixine or putaindicine, or microcin or subtilosin A), lanbiotics (including nisin, subtilin, epidermin, mutacin, mersacidin, actagardine, cinnamycin), lacticins and other antimicrobial or anti-inflammatory compounds.

In one aspect, a therapeutic composition used here comprises a reconstituted fecal flora consisting essentially of a combination of a purified fecal microbiota and a non-cellular fecal filtrate. In another aspect, a therapeutic composition used here comprises a purified fecal microbiota supplemented with one or more non-cellular non-particulate fecal components. In one aspect, a therapeutic composition used here comprises one or more non-cellular non-particulate fecal components. In one aspect, one or more non-cellular non-particulate fecal components comprise synthetic molecules, biologically active molecules produced by a fecal microorganism, or both. In another aspect, one or more non-cellular non-particulate fecal components comprise biologically active proteins or peptides, micronutrients, fats, sugars, small carbohydrates, trace elements, mineral salts, ash, mucous, amino acids, nutrients, vitamins, minerals, or any combination thereof. In one aspect, one or more non-cellular non-particulate fecal components comprise one or more biologically active molecules selected from the group consisting of bacteriocin, lanbiotic, and lacticin. In another aspect, one or more non-cellular non-particulate fecal components comprise one or more bacteriocins selected from the group consisting of colicin, troudulixine, putaindicine, microcin, and subtilosin A. In one aspect, one or more non-cellular non-particulate fecal components comprise one or more lanbiotics selected from the group consisting of thuricin, nisin, subtilin, epidermin, mutacin, mersacidin, actagardine, and cinnamycin. In another aspect, one or more non-cellular non-particulate fecal components comprise an anti-spore compound, an antimicrobial compound, an anti-inflammatory compound, or any combination thereof. In a further aspect, one or more non-cellular non-particulate fecal components comprise an interleukin, a cytokine, a leukotriene, an eicosanoid, or any combination thereof.

In another aspect, a treatment method provided here comprises the use of both fecal bacterial cells, e.g., a partial or a complete representation of the human GI microbiota, and an isolated, processed, filtered, concentrated, reconstituted and/or artificial liquid component (e.g., fecal filtrate) of the flora (the microbiota) which comprises, among others ingredients, bacterial secretory products such as e.g., bacteriocins (proteinaceous toxins produced by bacteria, including colicin, troudulixine or putaindicine, or microcin or subtilosin A), lanbiotics (a class of peptide antibiotics that contain a characteristic polycyclic thioether amino acid lanthionine or methyllanthionine, and unsaturated amino acids dehydroalanine and 2-aminoisobutyric acid; which include thuricin (which is secreted by bacilli in donor stools), nisin, subtilin, epidermin, mutacin, mersacidin, actagardine, cinnamycin), a lacticin (a family of pore-forming peptidic toxins) and other antimicrobial or anti-inflammatory compounds and/or additional biologically active molecules (BAMs) produced by bacteria or other microorganisms of the microbiota, and/or which are found in the “liquid component” of a microbiota.

In one aspect, a fecal bacteria-based therapeutic composition is used concurrently with a fecal non-cellular filtrate-based therapeutic composition. In another aspect, a patient is treated with a first fecal non-cellular filtrate-based therapeutic composition before being given a second fecal bacteria-based therapeutic composition, or vice versa. In a further aspect, a treatment method comprises three steps: first, antibiotic pre-treatment to non-selectively remove infectious pathogen(s); second, a fecal non-cellular filtrate-based treatment step to further suppress selected infectious pathogen(s); and third, giving the patient a fecal bacteria-based therapeutic composition to re-establish a functional intestinal microbiome.

In an aspect, a therapeutic composition is combined with other adjuvants such as antacids to dampen bacterial inactivation in the stomach. (e.g., Mylanta, Mucaine, Gastrogel). In another aspect, acid secretion in the stomach could also be pharmacologically suppressed using H2-antagonists or proton pump inhibitors. An example H2-antagonist is ranitidine. An example proton pump inhibitor is omeprazole. In one aspect, an acid suppressant is administered prior to administering, or in co-administration with, a therapeutic composition.

In an aspect, a therapeutic composition is in the form of: an enema composition which can be reconstituted with an appropriate diluent; enteric-coated capsules; enteric-coated microcapsules; acid-resistant tablet; acid-resistant capsules; acid-resistant microcapsules; powder for reconstitution with an appropriate diluent for naso-enteric infusion or colonoscopic infusion; powder for reconstitution with appropriate diluent, flavoring and gastric acid suppression agent for oral ingestion; powder for reconstitution with food or drink; or food or food supplement comprising enteric-coated and/or acid-resistant microcapsules of the composition, powder, jelly, or liquid.

In an aspect, a treatment method effects a cure, reduction of the symptoms, or a percentage reduction of symptoms of irritable bowel syndrome. The change of flora is preferably as “near-complete” as possible and the flora is replaced by viable organisms which will crowd out any remaining, original flora. Typically the change in enteric flora comprises introduction of an array of predetermined flora into the gastro-intestinal system, and thus in a preferred form the method of treatment comprises substantially or completely displacing pathogenic enteric flora in patients requiring such treatment.

In another aspect, a therapeutic composition can be provided together with a pharmaceutically acceptable carrier. As used herein, a “pharmaceutically acceptable carrier” refers to a non-toxic solvent, dispersant, excipient, adjuvant, or other material which is mixed with a live bacterium in order to permit the formation of a pharmaceutical composition, e.g., a dosage form capable of administration to the patient. A pharmaceutically acceptable carrier can be liquid (e.g., saline), gel or solid form of diluents, adjuvant, excipients or an acid resistant encapsulated ingredient. Suitable diluents and excipients include pharmaceutical grades of physiological saline, dextrose, glycerol, mannitol, lactose, starch, magnesium stearate, sodium saccharin, cellulose, magnesium carbonate, and the like, and combinations thereof. In another aspect, a therapeutic composition may contain auxiliary substances such as wetting or emulsifying agents, stabilizing or pH buffering agents. In an aspect, a therapeutic composition contains about 1%-5%, 5%-10%, 10%-15%, 15-20%, 20%-25%, 25-30%, 30-35%, 40-45%, 50%-55%, 1%-95%, 2%-95%, 5%-95%, 10%-95%, 15%-95%, 20%-95%, 25%-95%, 30%-95%, 35%-95%, 40%-95%, 45%-95%, 50%-95%, 55%-95%, 60%-95%, 65%-95%, 70%-95%, 45%-95%, 80%-95%, or 85%-95% of active ingredient. In an aspect, a therapeutic composition contains about 2%-70%, 5%-60%, 10%-50%, 15%-40%, 20%-30%, 25%-60%, 30%-60%, or 35%-60% of active ingredient.

In an aspect, a therapeutic composition can be incorporated into tablets, drenches, boluses, capsules or premixes. Formulation of these active ingredients into such dosage forms can be accomplished by means of methods well known in the pharmaceutical formulation arts. See, e.g., U.S. Pat. No. 4,394,377. Filling gelatin capsules with any desired form of the active ingredients readily produces capsules. If desired, these materials can be diluted with an inert powdered diluent, such as sugar, starch, powdered milk, purified crystalline cellulose, or the like to increase the volume for convenience of filling capsules.

In an aspect, conventional formulation processes can be used to prepare tablets containing a therapeutic composition. In addition to the active ingredients, tablets may contain a base, a disintegrator, an absorbent, a binder, and a lubricant. Typical bases include lactose, sugar, sodium chloride, starch and mannitol. Starch is also a good disintegrator as is alginic acid. Surface-active agents such as sodium lauryl sulfate and dioctyl sodium sulphosuccinate are also sometimes used. Commonly used absorbents include starch and lactose. Magnesium carbonate is also useful for oily substances. As a binder there can be used, for example, gelatin, gums, starch, dextrin, polyvinyl pyrrolidone and various cellulose derivatives. Among the commonly used lubricants are magnesium stearate, talc, paraffin wax, various metallic soaps, and polyethylene glycol.

In an aspect, for preparing solid compositions such as tablets, an active ingredient is mixed with a pharmaceutical carrier, e.g., conventional tableting ingredients such as corn starch, lactose, sucrose, sorbitol, talc, stearic acid, magnesium stearate, dicalcium phosphate or gums, or other pharmaceutical diluents, e.g. water, to form a solid preformulation composition containing a homogeneous mixture of a composition of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing a desired amount of an active ingredient (e.g., at least about 10⁵, 10⁶, 10⁷, 10⁸, 10⁹, 10¹⁰, 10¹¹, 10¹², or 10¹³ cfu). A therapeutic composition used herein can be flavored.

In an aspect, a therapeutic composition can be a tablet or a pill. In one aspect, a tablet or a pill can be coated or otherwise compounded to provide a dosage form affording the advantage of prolonged action. For example, a tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

In an aspect, a therapeutic composition can be a drench. In one aspect, a drench is prepared by choosing a saline-suspended form of a therapeutic composition. A water-soluble form of one ingredient can be used in conjunction with a water-insoluble form of the other by preparing a suspension of one with an aqueous solution of the other. Water-insoluble forms of either active ingredient may be prepared as a suspension or in some physiologically acceptable solvent such as polyethylene glycol. Suspensions of water-insoluble forms of either active ingredient can be prepared in oils such as peanut, corn, sesame oil or the like; in a glycol such as propylene glycol or a polyethylene glycol; or in water depending on the solubility of a particular active ingredient. Suitable physiologically acceptable adjuvants may be necessary in order to keep the active ingredients suspended. Adjuvants can include and be chosen from among the thickeners, such as carboxymethylcellulose, polyvinyl pyrrolidone, gelatin and the alginates. Surfactants generally will serve to suspend the active ingredients, particularly the fat-soluble propionate-enhancing compounds. Most useful for making suspensions in liquid nonsolvents are alkylphenol polyethylene oxide adducts, naphthalenesulfonates, alkylbenzene-sulfonates, and the polyoxyethylene sorbitan esters. In addition many substances, which affect the hydrophilicity, density and surface tension of the liquid, can assist in making suspensions in individual cases. For example, silicone anti-foams, glycols, sorbitol, and sugars can be useful suspending agents.

In an aspect, a therapeutic composition comprises non-pathogenic spores of one or more, two or more, three or more, or four or more Clostridium species selected from the group consisting of Clostridium absonum, Clostridium argentinense, Clostridium baratii, Clostridium botulinum, Clostridium cadaveris, Clostridium carnis, Clostridium celatum, Clostridium chauvoei, Clostridium clostridioforme, Clostridium cochlearium, Clostridium fallax, Clostridium felsineum, Clostridium ghonii, Clostridium glycolicum, Clostridium haemolyticum, Clostridium hastiforme, Clostridium histolyticum, Clostridium indolis, Clostridium irregulare, Clostridium limosum, Clostridium malenominatum, Clostridium novyi, Clostridium oroticum, Clostridium paraputrificum, Clostridium perfringens, Clostridium piliforme, Clostridium putrefaciens, Clostridium putrificum, Clostridium sardiniense, Clostridium sartagoforme, Clostridium scindens, Clostridium septicum, Clostridium sordellii, Clostridium sphenoides, Clostridium spiroforme, Clostridium sporogenes, Clostridium subterminale, Clostridium symbiosum, Clostridium tertium, Clostridium tetani, Clostridium welchii, and Clostridium villosum.

In an aspect, a therapeutic composition comprises purified, isolated, or cultured viable non-pathogenic Clostridium and a plurality of purified, isolated, or cultured viable non-pathogenic microorganisms from one or more genera selected from the group consisting of Collinsella, Coprococcus, Dorea, Eubacterium, and Ruminococcus. In another aspect, a therapeutic composition comprises a plurality of purified, isolated, or cultured viable non-pathogenic microorganisms from one or more genera selected from the group consisting of Clostridium, Collinsella, Coprococcus, Dorea, Eubacterium, and Ruminococcus.

In an aspect, a therapeutic composition comprises two or more genera selected from the group consisting of Collinsella, Coprococcus, Dorea, Eubacterium, and Ruminococcus. In another aspect, a therapeutic composition comprises two or more genera selected from the group consisting of Coprococcus, Dorea, Eubacterium, and Ruminococcus. In a further aspect, a therapeutic composition comprises one or more, two or more, three or more, four or more, or five or more species selected from the group consisting of Coprococcus catus, Coprococcus comes, Dorea longicatena, Eubacterium eligens, Eubacterium hadrum, Eubacterium hallii, Eubacterium rectale, and Ruminococcus torques.

In one aspect, a therapeutic composition comprises at least about 10⁵, 10⁶, 10⁷, 10⁸, 10⁹, 10¹⁰, 10¹¹, 10¹², or 10¹³ cfu or total cell count. In another aspect, a therapeutic composition comprises at most about 10⁵, 10⁶, 10⁷, 10⁸, 10⁹, 10¹⁰, 10¹¹, 10¹², 10¹³ or 10¹⁴ cfu or total cell count.

In another aspect, a therapeutic composition comprises at least about 10⁵, 10⁶, 10⁷, 10⁸, 10⁹, 10¹⁰, 10¹¹, 10¹², or 10¹³ cells or total cell count. In another aspect, a therapeutic composition comprises at most about 10⁵, 10⁶, 10⁷, 10⁸, 10⁹, 10¹⁰, 10¹¹, 10¹², 10¹³ or 10¹⁴ cells or total cell count.

In one aspect, a therapeutic composition is formulated as an oral capsule, microcapsule, tablet, or pill. In another aspect, a capsule, microcapsule, tablet, or pill is adapted for enteric delivery. In a further aspect, a capsule, microcapsule, tablet, or pill is an enteric capsule, microcapsule, tablet, or pill. In another aspect, a capsule, microcapsule, tablet, or pill comprises an enteric coating, is acid resistant, or both.

### EXAMPLES

### Example 1. Preparation of Fecal Microbiota

Fecal microbiota is prepared essentially according to protocols published in US2014/0147417 or WO2014/152484. Summarized below is an exemplary protocol.

Potential fecal microbiota donors are screened according to a list of criteria used to exclude unsuitable donors. Potential fecal microbiota donors are excluded if they have received antibiotics, laxatives, diet pills, immunomodulators or chemotherapy in the preceding three months. Potential fecal microbiota donors are excluded if they have a history of all known infectious diseases, morbid obesity, diabetes, irritable bowel syndrome, inflammatory bowel disease, chronic diarrhea, constipation, colorectal polyps or cancer, a compromised immune system, metabolic syndromes, chronic fatigue syndrome, major GI surgery, or other diseases or conditions potentially associated with specific changes in fecal microbiota. Potential fecal microbiota donors are excluded if they exhibit positive laboratory tests for C-reactive protein, erythrocyte sedimentation rate, hepatitis A, hepatitis B, hepatitis C, human immunodeficiency virus, human T-lymphotropic virus, or syphilis. Potential fecal microbiota donors are excluded if they exhibit a positive test for stool ova, parasites or viruses. Potential fecal microbiota donors are excluded if they engage in high-risk sexual behaviors, have been incarcerated, or received any tattoos or body piercings in areas that have had disease epidemics within the past three months.

Donor fecal material (fresh feces) is collected in a sterilized container, and then it is transferred to a blender. Approximately 500-1000 mL 0.9% saline solution is added to the blender and thoroughly mixed with the fecal sample. The resulting suspension is filtered at least 4 times through strainers prior to collecting a final suspension. The final suspension is centrifuged in 50 mL tubes at 1200×g for 3 minutes. The supernatant is discarded and the pellet is gently resuspended in approximately 50 mL of sterile 0.9% saline solution. The centrifugation and resuspension steps are repeated 2 to 4 additional times. Upon the final centrifugation, the supernatant is discarded. If the fecal microbiota is to be used immediately, the resultant pellet is resuspended in 1.5-volumes of 0.9% saline solution by gently mixing. If the fecal microbiota is to be stored, the resultant pellet is resuspended in 10% sterile glycerol and stored at −80 degrees Centigrade. If fecal microbiota are frozen, they are warmed to room temperature prior to administration to a patient.

### Example 2. Treatment of Irritable Bowel Syndrome with Diarrhea (IBS-D)

A 40-year old male patient with symptoms of irritable bowel syndrome with diarrhea for greater than 3 years is treated with fecal microbiome therapy. The patient's symptoms include diarrhea, nausea, bloating, joint pain, tenesmus, malaise, lethargy, feeling of incomplete evacuation, dairy intolerance and abdominal discomfort. The fecal microbiome therapy regimen includes transcolonoscopic infusion and nine subsequent enema infusions. Three months post-fecal microbiome therapy the patient shows a 75% improvement in bloating, tenesmus, feeling of incomplete evacuation, abdominal discomfort, nausea, joint pain, and lethargy. The patient also experiences complete resolution of diarrhea and is no longer dairy intolerant.

### Example 3. Treatment of Irritable Bowel Syndrome with Diarrhea (IBS-D)

A 49-year old female with symptoms of irritable bowel syndrome with diarrhea (IBS-D) for greater than 4 years is treated with fecal microbiome therapy. The patient's symptoms include diarrhea, flatulence, abdominal pain, decreased mental acuity, daily nausea, and extensive food intolerances. The patient is administered an antibiotic pretreatment comprising oral vancomycin (250 mg mane, 500 mg nocte) and oral rifaximin (200 mg twice daily) for 3 months prior to fecal microbiome therapy. The fecal microbiome therapy regimen includes transcolonoscopic infusion and nine subsequent enema infusions. Five months post-fecal microbiome therapy the patient shows a 90% resolution of diarrhea and experiences just 1 stool/day. The patient also experiences improvement in food tolerances and a reduction in bloating.

### Example 4. Treatment of Irritable Bowel Syndrome with Diarrhea (IBS-D)

A 78-year old male with symptoms of irritable bowel syndrome with diarrhea (IBS-D) for 5 years was treated with fecal microbiome therapy. The patient's symptoms include diarrhea with 2-3 watery motions/day. The patient is administered an antibiotic pretreatment of oral vancomycin (500 mg twice daily) for 12 months prior to fecal microbiome therapy. The fecal microbiome therapy regimen includes transcolonoscopic infusion and 4 enema infusions. Three months post-fecal microbiome therapy the patient shows resolution of diarrhea with 1-2 soft, formed motions/day.

### Example 5. Treatment of Irritable Bowel Syndrome with Diarrhea (IBS-D)

A 51-year old female with symptoms of IBS-D for 12 years is treated with FMT. The patient's symptoms include 1-12 watery motions/day, abdominal pain, flatulence, bloating, fatigue, and decreased mental acuity. The fecal microbiome therapy regimen includes transcolonoscopic infusion and nine subsequent enema infusions. Six months post-fecal microbiome therapy the patient shows resolution of diarrhea with 2-3 formed motions/day. The patient also experiences infrequent abdominal pain and increased energy.

### Example 6. Treatment of Irritable Bowel Syndrome with Diarrhea (IBS-D)

A 29-year old male with symptoms of IBS-D for 3 years is treated with fecal microbiome therapy. The patient's symptoms include diarrhea with 5 motions/day, abdominal cramping and pain, severe urgency, and 10 kg weight loss. The patient is prescribed Loperamide for symptom control. The patient was administered an antibiotic pretreatment of oral vancomycin (500 mg twice daily) and oral metronidazole (200 mg twice daily) for 1 month prior to fecal microbiome therapy. The fecal microbiome therapy regimen includes transcolonoscopic infusion and nine subsequent enema infusions. Six months post-fecal microbiome therapy the patient shows resolution of diarrhea with occasional episodic diarrhea and generally 1-2 motions/day. The patient also experiences minimal pain, a resolution of bloating, and a cessation of Loperamide medication.

### Example 7. Treatment of Irritable Bowel Syndrome with Diarrhea (IBS-D)

A 58-year old female with symptoms of IBS-D for greater than 28 years is treated with fecal microbiome therapy. The patient's symptoms include explosive diarrhea with up to 7 motions/day, the patient is administered an antibiotic pretreatment of oral vancomycin (500 mg twice daily) for two months prior to fecal microbiome therapy. The fecal microbiome therapy regimen includes TC infusion and four subsequent enema infusions. Nine months post-fecal microbiome therapy the patient shows increased food tolerance, cessation of Loperamide and cholestyramine medications previously used to control diarrhea. The patient experiences 1-2 formed motions/day.

### Example 8. Treatment of Irritable Bowel Syndrome with Diarrhea (IBS-D)

A 60-year old male with symptoms of IBS-D for greater than 15 years is treated with fecal microbiome therapy. The patient's symptoms include long-standing diarrhea with more than 10 watery motions/day, severe abdominal pain, cramping, bloating, nausea, and urgency. The patient is administered an antibiotic pretreatment of oral vancomycin (250 mg twice daily) and rifaximin (200 mg twice daily) for two months prior to fecal microbiome therapy. The fecal microbiome therapy regimen includes transcolonoscopic infusion and four subsequent enema infusions. Ten months post-fecal microbiome therapy the patient shows improvement with only intermittent symptoms of episodic diarrhea with colicky pain.

### Example 9. Treatment of Multiple Sclerosis and Irritable Bowel Syndrome (IBS)

A 39-year old male patient, with longstanding irritable bowel syndrome and multiple sclerosis, that has been dormant and stable for two years, is treated with fecal microbiome therapy. The patient's symptoms include muscle fatigue, loss of balance, walking difficulty with much weakness and require support, and slurred speech. The patient also shows demyelination on Mill which later improves on a repeat Mill.

The patient receives fecal microbiome therapy through the colonoscope into the ascending colon, followed by nine enemas over the next week to increase the bacterial load. The patient has an initial great response for two months and is symptom free from both IBS and MS. The patient's energy returns and there are no reports of nausea. Further, the patient passes two formed motions per day, and is able to walk without support after treatment. This marks the only period of good health that the patient has for many years. However, after two months of improvement, his symptoms begin recurring and he reverts to where his colonic flora was prior to fecal microbiome therapy. The patient is prescribed Rifaximin and low dose probiotics, which has little effect on his multiple sclerosis and IBS symptoms.

### Example 10. Treatment of Irritable Bowel Syndrome with Diarrhea (IBS-D)

A 78-year old female patient with symptoms of irritable bowel syndrome with diarrhea for greater than 10 years is treated with fecal microbiome therapy. The patient's symptoms include inconsistence, nocturnal stools, pain in left side, urgency, and chronic diarrhea with 4-6 stools/day. The patient is prescribed 10 mg loperamide daily. The patient is administered an antibiotic pretreatment comprising oral vancomycin (500 mg twice daily) and oral metronidazole (200-400 mg twice daily) for 9 months prior to fecal microbiome therapy. The fecal microbiome therapy regimen includes transcolonoscopic infusion and one enema infusion. Three months post-fecal microbiome therapy the patient's symptoms decreased to occasional diarrhea and approximately 2 stools/day.

### Example 11. Treatment of Irritable Bowel Syndrome with Diarrhea (IBS-D)

A 79-year old patient with symptoms of irritable bowel syndrome is treated with acid resistant/delayed release oral capsules containing lyophilized donor-derived non-selected fecal microbiota. Each capsule contains a total cell count of about 10⁹-10¹². Similar capsules are used for Examples 11 to 16. The patient's symptoms include severe diarrhea with 8-10 stools/day (with stool consistency of 6-7 on the BRISTOL scale), moderate bloating, severe abdominal discomfort, severe urgency, increased flatulence, brain fog, general malaise, joint pain, loss of appetite, weight loss, and nausea. The patient is administered an induction treatment 144 capsules (across a treatment duration of 12 weeks). The patient receives 50 capsules in week 1 (day 1-5: 2 qid; day 6: tid; day 7: 2 qd); 28 in week 2 (2 bid); in week 3-4 (2 qd); week 5-6 (1 qd); week 7-12: 1 qod). At week 12 of the 12-week treatment schedule, the patient's symptoms decrease to diarrhea 4-8 stools/day, decreased urgency, no abdominal pain, no bloating, decreased flatulence, mild malaise, and mild nausea. The patient's brain fog remains and headaches increase.

### Example 12. Treatment of Irritable Bowel Syndrome with Diarrhea (IBS-D)

A 74-year old patient with symptoms of irritable bowel syndrome is treated with acid resistant/delayed release oral capsules containing lyophilized donor-derived non-selected fecal microbiota. The patient's symptoms include severe diarrhea with 15 stools/day, intermittent bloating, severe abdominal discomfort, and severe urgency. The patient is subject to a 6-week treatment schedule which starts with an induction treatment of one colonoscopic fecal microbiome therapy liquid infusion and ten fecal microbiome therapy liquid rectal enema infusions within two weeks and capsule maintenance treatment of 1 bid for four weeks. At week 6 of the 6-week treatment schedule, the patient's symptoms decrease to diarrhea 8-9 stools/day. Urgency, bloating, and abdominal discomfort remain. The patient dosing schedule is changed to one capsule/day. The patient is also prescribed movicol as needed during travel.

### Example 13. Treatment of Irritable Bowel Syndrome with Constipation (IBS-C)

A 26-year old patient with symptoms of irritable bowel syndrome is treated with acid resistant/delayed release oral capsules containing lyophilized donor-derived non-selected fecal microbiota. The patient's symptoms include mild to severe constipation with 0-1 stools/day, bloating, abdominal discomfort, urgency present but fails to open bowels, chronic fatigue, nausea, headaches, brain fog, inability to concentrate, and poor outlook on life. The patient is subject to a 12-week treatment schedule which includes an induction treatment of 138 capsules over a period of 12 weeks (week 1, day 1-5: 2 qid; day 6-7: 2 bid; week 2: 2 bid; week 3-4: 2 qd; week 5-6: 1 qd; week 7-12: 1 qod) and a maintenance treatment of two capsules/day for four weeks. At week 6 of the scheduled treatment, the patient's symptoms of constipation improve to 1-2 stools/day. The symptoms of urgency, fatigue, general malaise, and flatulence are all mild. Bloating and abdominal discomfort no longer persists. By week 12 the patient has no fatigue, a major improvement in energy levels, can run a marathon, has a great outlook on life, and shows none of the previously presented symptoms.

### Example 14. Treatment of Irritable Bowel Syndrome with Constipation (IBS-C)

A 28-year old patient with symptoms of irritable bowel syndrome is treated with acid resistant/delayed release oral capsules containing lyophilized donor-derived non-selected fecal microbiota. The patient's symptoms include mild to severe constipation with 1 stool/day, moderate pain upon defecation, severe bloating, severe urgency, severe abdominal pain and discomfort, increased flatulence, brain fog, headaches, general malaise, loss of appetite, nausea, and food allergies. The patient is subject to a 12-week treatment schedule which includes an induction treatment of 142 capsules over a period of 12 weeks (week 1, day 1-5: 2 qid; day 6-7: 2 bid; week 2: 2 bid; week 3-4: 2 qd; week 5-6: 1 qd; week 7-12: 1 qod). At week 8 of the scheduled treatment, the patient's symptoms of constipation improve to 3-4 stools/day. The symptoms of bloating and abdominal discomfort are severe, and urgency decreases. The patient continues to experience symptoms of flatulence, brain fog, loss of appetite, nausea, food allergies, and headaches. The patient ceases treatment early at week 11, hence only subjected to 138 capsules over 11 weeks, at which time the patient has no significant overall symptom improvement compared to at the start of the treatment.

### Example 15. Treatment of Irritable Bowel Syndrome with Constipation (IBS-C)

A 11-year old patient with symptoms of irritable bowel syndrome is treated with acid resistant/delayed release oral capsules containing lyophilized donor-derived non-selected fecal microbiota. The patient's symptoms include severe constipation with 1 stool/day, moderate pain upon defecation, mild urgency, and moderate abdominal discomfort, chronic fatigue, lack of concentration (inhibiting the patient from attending school), severe headaches, brain fog, leg aches, loss of appetite, nausea, and food allergies. The patient is positive for Clostridium difficile and Clostridium perfringens. The patient is subject to a first 12-week treatment schedule which includes an induction treatment of 178 capsules. Patient receives no maintenance treatment. At week 2 of the scheduled treatment, the patient's symptoms of constipation remain (1-2 stools/day). The additional symptoms are improved by about 40% according to the patient. The patient is negative for Clostridium difficile and Clostridium perfringens. The patient ceases treatment at week six of the planned 12 week treatment.

The patient subsequently enters a second treatment with double encapsulated capsules. Prior to beginning the second treatment, the patient experiences severe constipation with 1 stool/day or 1 stool/every 2 days with medication (dipentum and colgout). The patient's symptoms also include severe urgency (after dipentum and colgout), severe abdominal discomfort, chronic fatigue, lack of concentration (inhibiting the patient from attending school), severe headaches, brain fog, severe leg aches, loss of appetite, nausea, mild fever and food allergies. The patient is subject to a 10-week treatment schedule and is administered an induction treatment of 294 capsules (week 1-2: 2 qid; week 3-4: 2 tid; week 5-6: 2 bid; week 7-10: 2 qd). At weeks two and four, the patient's constipation does not improve. The patient's abdominal discomfort improves to moderate and urgency ceases. The patient is negative for Clostridium difficile and Clostridium perfringens. The patient attends school for 1 hour/day, remains in bed for 22 hours, and has one half hour of outside play. Between week 6 and week 9, the patient only experiences mild abdominal discomfort with no urgency. At week 6, the patient also exhibit increased energy levels and improved appetite. Energy levels later decrease at weeks 8 and 9 potentially due to the patient's weaning off of capsules. Thereafter patient is subjected to maintenance capsules (2 bid) for four weeks. The maintenance capsules keep him stable by an overall 40% improvement.

### Example 16. Treatment of Irritable Bowel Syndrome with Constipation (IBS-C) and Cyclic Vomiting Syndrome (CVS)

A 30-year old patient with symptoms of irritable bowel syndrome is treated with acid resistant/delayed release oral capsules containing lyophilized donor-derived non-selected fecal microbiota. The patient's symptoms include severe constipation with 0 stool/day, severe urgency, severe bloating, severe abdominal discomfort, recurrent daily vomiting episodes, mild headaches, mucus in stool, nausea, and food allergies. The patient is administered an induction treatment of 136 capsules over a period of 10 weeks (day 1-5: 2 qid; day 6: tid; day 7: 2 qd; 28 in week 2: 2 bid; in week 3-4:2 qd; week 5-10: 1 qd). Patient does not participate in maintenance treatment. At week 10 post-initial capsule treatment, the patient's symptoms of constipation are improved (0-1 stools/day). Vomiting is improved to once every two weeks. The patient experiences mild headaches, some food allergies, no mucus in stool, and no nausea.

### Example 17. Orally-Administered Lyophilised Full-Spectrum Microbiota for the Treatment of Irritable Bowel Syndrome with Constipation with Dose C1 (IBS-C)

Patients are divided into four groups (Groups 1 to 4). Group 1 patients are administered a pre-treatment of antibiotics (e.g., Vancomycin, Metronidazole, and Colchicine) until the patient shows signs of improved bowel movements. Group 2 receives no antibiotics. Both Groups 1 and 2 receive a pre-colonoscopy bowel prep followed by capsule fecal microbiome therapy. Groups 3 and 4 receive no bowel prep while Group 3, not group 4, also receive an antibiotic pretreatment. Capsules are administered for 18 weeks as follows: two capsules twice-a-day for 14 days, two capsules twice-a-day every other day for 14 days, 4 capsules twice-a-week for 14 days, and 4 capsules once-a-week (e.g., each Monday) for 12 weeks. High dose capsules (total cell count of about 10¹²) are used in loading doses (also called treatment doses) for the initial 4 weeks. Lower dose capsules (total cell count of about 10⁹) are used in maintenance doses for the subsequent 14 weeks. In patients receiving antibiotic pretreatment, capsules are administered one day after ceasing antibiotics. Patient symptoms are observed and clinical examination is performed before, during and post oral capsule treatment. Pre, during and post-treatment DNA metagenomics (2-4 days; 1 week; 6 weeks; 12 weeks) are also carried out. The capsule treatments reverse patient symptoms and result in a clinically normal urge and defecation.

### Example 18. Orally-Administered Lyophilised Full-Spectrum Microbiota for the Treatment of Irritable Bowel Syndrome with Diarrhea (IBS-D)

Patients are divided into four groups (Groups 1 to 4). Group 1 patients are administered a pre-treatment of antibiotics (e.g., Vancomycin and Metronidazole) until the patient shows signs of improved bowel movements. Group 2 receives no antibiotics. Both Groups 1 and 2 receive a pre-colonoscopy bowel prep followed by capsule fecal microbiome therapy. Groups 3 and 4 receive no bowel prep while Group 3, but not Group 4, also receive an antibiotic pretreatment. Capsules are administered for 18 weeks or more as follows: two capsules twice-a-day for 14 days, two capsules twice-a-day every other day for 14 days, 4 capsules twice-a-week for 14 days, and 4 capsules once-a-week (e.g., each Monday) for 12 weeks. High dose capsules (total cell count of about 10¹²) are used in loading doses (also called treatment doses) for the initial 4 weeks. Lower dose capsules (total cell count of about 10⁹) are used in maintenance doses for the subsequent 14 weeks. In patients receiving antibiotic pretreatment, capsules are administered one day after ceasing antibiotics. Patient symptoms are observed and clinical examination is performed before, during and post oral capsule treatment. Pre, during and post-treatment DNA metagenomics (2-4 days; 1 week; 6 weeks; 12 weeks) are also carried out. The capsule treatments reverse patient symptoms and result in a clinically normal urge and defecation.

As various modifications could be made in the constructions and methods herein described and illustrated without departing from the scope of the disclosure, it is intended that all matter contained in the foregoing description shall be interpreted as illustrative rather than limiting. The breadth and scope of the present disclosure should not be limited by any of the above-described exemplary embodiments, but should be defined only in accordance with the following claims appended hereto and their equivalents. All patent and non-patent documents cited in this specification are incorporated herein by reference in their entirety.

1. A method for treating irritable bowel syndrome in a human subject in need thereof, said method comprising orally administering to said human subject a pharmaceutically active dose of a therapeutic composition comprising a capsule comprising non-selected fecal microbiota.

2.-91. (canceled)

92. The method of claim 1, wherein said irritable bowel syndrome is selected from the group consisting of irritable bowel syndrome with constipation, irritable bowel syndrome with diarrhea, and irritable bowel syndrome mixed type.

93. The method of claim 1, wherein said fecal microbiota comprises a donor's entire fecal microbiota.

94. The method of claim 1, wherein said method reduces the irritable bowel syndrome symptom severity score (IBS-SSS) by at least 20% after at least 8 weeks of treatment.

95. The method of claim 1, wherein said dose is administered at least once daily for at least 2 weeks.

96. The method of claim 1, wherein said dose is administered at least twice weekly for at least two weeks.

97. The method of claim 96, wherein said dose is administered at least twice weekly for at least 4 weeks.

98. The method of claim 96, wherein said dose is administered at least twice weekly for at least 8 weeks.

99. The method of claim 1, wherein said dose is administered at least three times weekly for at least 4 weeks.

100. The method of claim 1, wherein said therapeutic composition comprises both live non-pathogenic fecal bacteria and a non-cellular fecal filtrate.

101. The method of claim 1, wherein said method comprises a first dosing schedule followed by a second dosing schedule, wherein said second dosing schedule comprises a maintenance dose lower or equal to the dose of said first dosing schedule.

102. The method of claim 1, wherein said therapeutic composition is formulated as a delayed or gradual enteric release form.

103. The method of claim 1, wherein said therapeutic composition is formulated as an enteric coated capsule or an acid-resistant capsule.

104. The method of claim 1, wherein said pharmaceutically active dose is from 10⁸ to 10¹⁴ cfu or total number of cells.

105. The method of claim 1, wherein said therapeutic composition comprises a cryoprotectant selected from the group consisting of polyethylene glycol, skim milk, erythritol, arabitol, sorbitol, glucose, fructose, alanine, glycine, proline, sucrose, lactose, ribose, trehalose, dimethyl sulfoxide (DMSO), glycerol, and a combination thereof.

106. The method of claim 1, wherein said subject is pretreated with an antibiotic prior to administration of said composition.

107. The method of claim 1, wherein said method eliminates or reduces one or more irritable bowel syndrome symptoms selected from the group consisting of abdominal cramping, abdominal pain, bloating, gas, diarrhea, constipation, hard stool, dry stool, mucus in the stool, depression, anxiety, fatigue, fibromyalgia, sleep disturbances, and chronic headaches.

108. The method of claim 1, wherein said therapeutic composition is in a liquid, frozen, freeze-dried, spray-dried, foam-dried, or powder form.

109. The method of claim 1, wherein said non-selected fecal microbiota consist essentially of spores.

110. The method of claim 1, wherein said non-selected fecal microbiota comprises Bifidobacterium.

111. A method for treating irritable bowel syndrome in a human subject in need thereof, said method comprising orally administering to said human subject a pharmaceutically active dose of a freeze-dried fecal microbiota composition comprising Bifidobacterium.

112. The method of claim 111, wherein said irritable bowel syndrome is selected from the group consisting of irritable bowel syndrome with constipation, irritable bowel syndrome with diarrhea, and irritable bowel syndrome mixed type.

113. The method of claim 111, wherein said active dose is administered at least once daily for at least 2 weeks.

114. The method of claim 111, wherein said active dose is administered at least twice weekly for at least 4 weeks.

115. The method of claim 111, wherein said composition comprises both live non-pathogenic fecal bacteria and a non-cellular fecal filtrate.

116. The method of claim 111, wherein said composition is formulated as a capsule.

117. The method of claim 116, wherein said capsule is an enteric coated capsule or an acid-resistant capsule.

118. The method of claim 111, wherein said freeze-dried fecal microbiota composition consists essentially of spores.

119. The method of claim 111, wherein said freeze-dried fecal microbiota composition is non-selected.

120. The method of claim 111, wherein said method eliminates or reduces one or more irritable bowel syndrome symptoms selected from the group consisting of abdominal cramping, abdominal pain, bloating, gas, diarrhea, constipation, hard stool, dry stool, mucus in the stool, depression, anxiety, fatigue, fibromyalgia, sleep disturbances, and chronic headaches.
